(19) United States
(12) Patent Application Publication
(10) Pub. No.: US 2006/0041190 A1
(43) Pub. Date: Feb. 23, 2006

(54) Operation button for endoscope

(75) Inventor: Yasuyuki Sato, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/206,996

(22) Filed: Aug. 19, 2005

(30) Foreign Application Priority Data
Aug. 20, 2004 (JP) 2004-241620

(51) Int. Cl. A61B 1/12

(52) U.S. Cl. 600/159

(57) ABSTRACT

An operation button for an endoscope is provided with a cylinder and a piston reciprocally slidable inside a lumen of the cylinder. A first flow channel and a second flow channel are formed to communicate with the lumen. When the piston is located at a first position, the first and second flow channel communicate with each other, while the first flow channel and the second flow channel are disconnected when the piston is located at a second position. A sealing member is provided on an outer periphery of the piston to provide secure airtightness between the cylinder and the piston. The sealing member includes a core member mainly made of one of poly-para-xylylene and poly-para-xylylene derivatives and a coating layer allocated on an outer periphery of the core member.

### BACKGROUND OF THE INVENTION

The present invention relates to an operation button for an endoscope.

An endoscope is configured to have several features for operations such as to aspirate liquid, for example body fluid or blood from body cavities, and to provide certain gas or fluid into body cavities. These operations are generally carried out by pressing a button (an operation button) that is equipped to an endoscope.

Such a button equipped to an endoscope is configured with a cylinder, a piston that is installed to reciprocally slide inside the cylinder, and an O-ring for maintaining airtightness between the cylinder and the piston. An example of such a structure is disclosed in Japanese Patent Provisional Publication No. 2000-271067.

Generally, an O-ring is made of an elastic material. If such a material has large friction resistance, lubricant such as silicone oil is applied to the surface of the O-ring.

However, according to the conventional structure, the lubricant should be applied repetitively to the surface of the O-ring after every certain period of time, which may be troublesome and inconvenient. In addition, too much amount of lubricant applied may leak to other connecting tubes and consequentially narrow the tubes.

### SUMMARY OF THE INVENTION

The present invention is advantageous in that an operation button provided with a slidable piston member and requiring substantially no lubricant is provided.

According to an aspect of the present invention, there is provided with an operation button for an endoscope that is provided with a cylinder that is provided with a lumen, and a first flow channel and a second flow channel that are communicating with the lumen, a piston that is installed inside the lumen of the cylinder to reciprocally slide between a first position, whereat the first flow channel is prevented from communicating with the second flow channel, and a second position, whereat the first flow channel is allowed to communicate with the second flow channel, and a sealing member that is provided with at an outer periphery of the piston, to obtain secure airtightness between the cylinder and the piston. The sealing member is configured with a core member made of an elastic material, and a coating layer, which is mainly made of one of poly-para-xylylene and poly-para-xylylene derivatives, that is allocated at an outer periphery of the core member and in immediate contact with the inner diameter of the cylinder.

Optionally, the polymerization degree of the one of the poly-para-xylylene and the poly-para-xylylene derivatives is greater than 5,000.

Optionally, there is provided an operation button for an endoscope wherein the elastic material is mainly made of silicone rubber.

Optionally, there is provided an operation button for an endoscope wherein an average thickness of the coating layer is in a range from 1 to 10 micrometers.

Optionally, there is provided an operation button for an endoscope wherein the piston has a penetrative port, which is configured such that the penetrative port does not connect the first flow channel with the second flow channel when the piston is in the first position, wherein the penetrative port allows the first flow channel to communicate with the second flow channel through the penetrative port.

Optionally, there is provided an operation button for an endoscope wherein the cylinder and the piston are slidable with each other with requiring substantially no lubricant.

According to another aspect of the invention, an endoscope including at least one operation button for an endoscope described above is provided.

### BRIEF DESCRIPTION OF THE ACCOMPANYING DRAWINGS

FIG. 1 is an illustrative view of an entire arrangement of an endoscope, in accordance with an exemplary embodiment of this invention.

FIG. 2 is a vertical cross-sectional view of an aspiration button provided to an endoscope shown in FIG. 1 in its ready position (the first position).

FIG. 3 is a vertical cross-sectional view of an aspiration button provided to an endoscope shown in FIG. 1 in its operation position (the second position).

FIG. 4 shows the measured results of the suppress strength in the comparative examples using O-rings.

### DETAILED DESCRIPTION OF THE EMBODIMENT

### First Embodiment

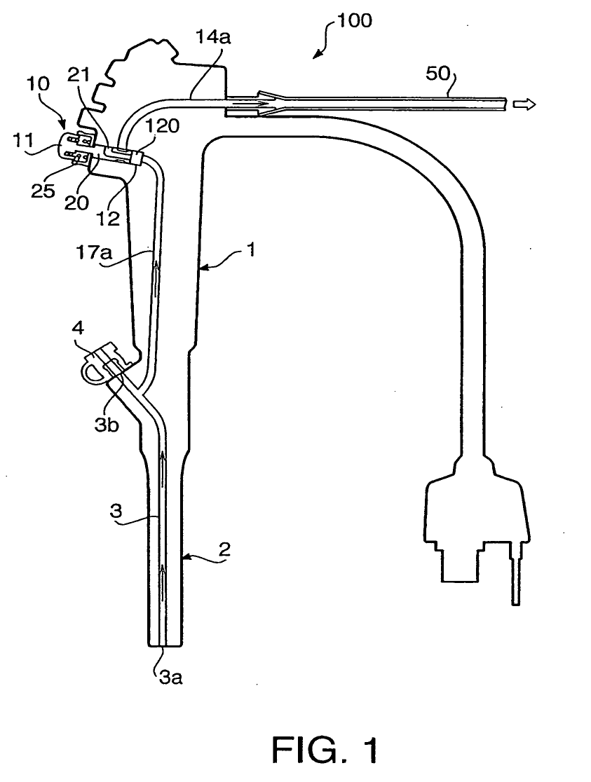

Referring to the accompanying drawings, an embodiment of the present invention will be described. In the following description, the lower end of each section shown in FIG. 1 is referred as distal end, while the upper end of each section is referred as proximal end.

FIG. 1 shows an endoscope 100 including an operation section 1, which is to be held by hand for operation of the entire endoscope 100, and an insertion section 2, which is provided at the distal end of the operation section 1 to be inserted into a cavity of a tubular organ.

The insertion section 2 is provided with a channel tube 3 penetrating through the inside thereof. The distal end 3a of the channel tube 3 (i.e., at the distal end of the insertion section 2) is provided with an opening. On the other end, a proximal end portion 3b is fixed in the vicinity to the connecting point of the operation section 1 and the insertion section 2, and a clamp stopper 4 is equipped thereto.

An aspiration button 10 provided on the operation section 1, includes a cylinder 12 having a lumen 120, a piston 20 that is installed inside the lumen 120 of the cylinder 12 to reciprocally slide in the axial direction, a recoverable spring 25 that applies an outward expanding force to the piston 20, and a press section 11 that is provided at the end of the piston 20, which protrudes outside the operation section 1.

The aspiration button 10 is configured such that, by pressing the press section 11, the piston 20 is moved toward the distal end of the cylinder 12.

The cylinder 12 is fixed to the proximal end portion of the operation section 1 by a nut 13 with an opening thereof facing to the operation section 1.

A lateral portion of the cylinder 12 is provided with an aspiration opening 14, whereto one end of an aspirator channel 14a (a first flow channel) is connected and communicates with the lumen 120. The other end of the aspirator channel 14a is connected to an exterior aspiration tube 50, which is communicating with an exterior aspiration unit (not shown).

Further, at one end of the cylinder 12 that is nearer to the proximal end portion of the operation section 1 is provided with a mutual opening 17, whereto one end of a connecting tube 17a is connected and communicating with the lumen 120. The other end of the connecting tube 17a is connected in the vicinity to the proximal end of the channel tube 3.

A lateral portion of the piston 20 is provided with a locator pin 20a protruding sideward. In addition, inside the cylinder 12 a linear slot 12a, which is dented along the axial direction thereof, is provided. This locator pin 20a and the slot 12a are provided to engage with each other, so that the piston 20 may not rotate inside the cylinder 12 and is located properly.

At an outer periphery of the piston 20, a piston cradle 22 is provided to surround a protruding portion of the piston 20. Further at an outer periphery of the piston cradle 22, an elastically deformative cover 22a, which is provided with an engaging portion to hold the nut 13, is integrally formed.

On the opposite end of the piston 20 from the cylinder 20, a mount 24 is provided to screw the press section 11 and the piston 20 together.

In between the mount 24 and the bottom of the piston cradle 22, a compressed recoverable spring 25, configured with a compression coil spring, is provided. Further, the center of the upper press section 11 is provided with an index 26 concentrically to the axis.

With this structure, the piston 20 and the press section 11 are integrated, and are steadily applied with an outward expanding force (in a direction away from the opening of the cylinder 12).

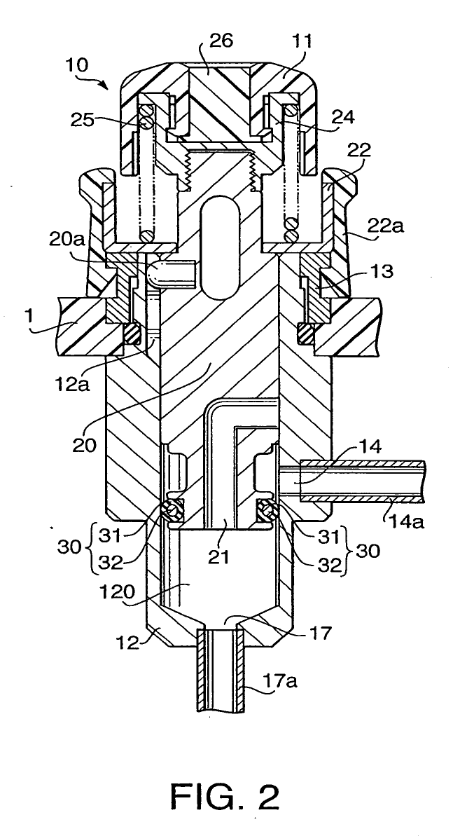

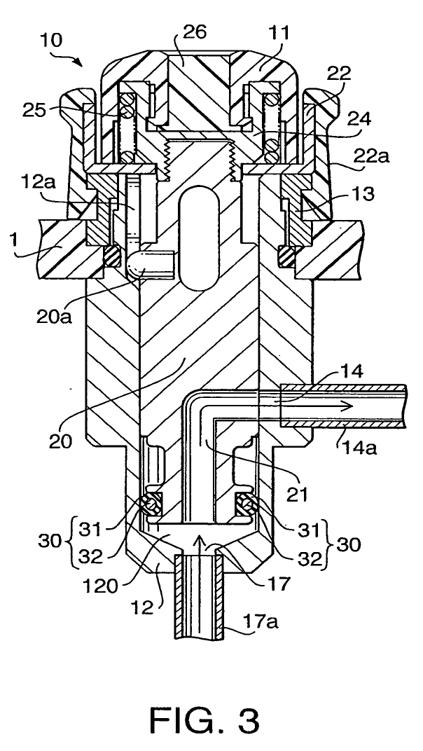

Inside the piston 20, on one end nearer to the proximal end of the connecting tube 17, is formed an L-shaped penetrative port 21. One opening of this penetrative port 21 is provided on the bottom end surface of the piston 20, while the other opening is provided on a lateral portion of the piston 20. When the piston 20 is in the first position, which is for example shown in FIG. 2, the aspirator channel 14a (a first flow channel) and the connecting tube 17a are in a state wherein they are not allowed to communicate with each other (hereinafter referred as “ready state”). On the other hand, when the piston 20 is in the second position, which is for example shown in FIG. 3, the aspirator channel 14a (a first flow channel) and the connecting tube 17a (a second flow channel) are in a state wherein they are allowed to communicate with each other via the penetrative port 21 (hereinafter referred as “operation state”).

At the outer periphery of the piston 20 and in the vicinity to the bottom is provided with a sealing member 30. This sealing member 30 is in immediate contact with the inside (inner periphery) of the cylinder 20 and provides secure airtightness between the cylinder 12 and the piston 20.

With the aspiration button 10 as described above, when the press section 11 is pressed inward, the aspirator channel 14a and the connecting tube 17a are communicated via the penetrative port 21, which allows fluid, for example body fluid or blood, to be aspirated in a direction from the connecting tube 17a toward the aspirator channel 14a through the channel tube 3. In addition, when the press section 11 is released, the aspirator channel 14a and the connecting tube 17a are immediately disconnected, which stops aspiration of the fluid via the channel tube 3.

In the present embodiment, the component of the sealing member 30 is provided with features including those described below.

The sealing member 30, as shown in FIG. 2 and FIG. 3, is configured with a core member 31 made of an elastic material, and a coating layer 32 that is allocated at the outer periphery of the core member 31 and in immediate contact with the inner periphery of the cylinder 12.

An elastic material made of the sealing member 30 may be one of various rubber materials, for example, silicone rubber, isoprene rubber, butadiene rubber, styrene-butadiene rubber, nitrile rubber, chloroprene rubber, butyl rubber, acrylic rubber, ethylene-propylene rubber, epichlorhydrin rubber, polyurethane rubber, fluorocarbon rubber, natural rubber, or one or combination of two or more materials such as styrene, polyolefin, polyvinyl chloride, polyurethane, polyamid, polybutadiene, transpolyisoprene, fluorocarbon rubber, chlorinated polyethylene. However, among all, it is preferable that the core member 31 of the sealing member 30 is mainly made of silicone rubber. With this configuration, elasticity of the sealing member 30 particularly increases, and airtightness between the cylinder 12 and the piston 20 is securely maintained.

In addition, silicone rubber provides high compatibility with a component of the coating layer, which will be described later, therefore adhesion between the core member 31 and the coating layer 32 is enhanced.

The coating layer 32 includes poly-para-xylylene and poly-para-xylylene derivatives (e.g. halogenide) as a main component. With this configuration, sealing tightness of the sealing member 30 with the inner periphery of the cylinder 12 is obtained, while the slide efficiency between the piston 20 and the cylinder 12 is improved. As a result, transition from the ready state to the operation state and vice versa by pressing or releasing the aspiration button 10 is accomplished smoothly and properly. More specifically, the operation efficiency of the aspiration button 10 is improved.

For other sealing members found in the prior art, lubricant such as silicone oil is applied to the surface thereof to obtain slide efficiency. In this case, however, such lubricant requires to be applied repetitively to the surface after every certain period of time, which may be troublesome and inconvenient. In addition, too much amount of lubricant applied may leak to other connecting tubes and consequentially narrow the tubes.

In regard to the present invention, however, a process to apply lubricant to the surface of the sealing member 30 can be eliminated, as the sealing member 30 has the coating layer 32 with higher slide efficiency. In other words, the cylinder 12 and the piston 20 are slidable with each other with requiring substantially no lubricant. Thus, a possible inconvenient process to repetitively apply lubricant to the surface of the sealing member 30 can be eliminated, and a trouble of narrowing in tubes caused by too much amount of lubricant can be explicitly avoided.

It is preferable, but not limited, that the polymerization degree (i.e. repeating unit) of the one of the poly-para-xylylene and the poly-para-xylylene derivatives is greater than 5,000. With this configuration, sealing tightness of the sealing member 30 with the inner periphery of the cylinder 12 is prevented from decaying, at the same time the slide efficiency between the piston 20 and the cylinder 12 is improved.

Further, it is preferable, but not limited, that an average thickness of the coating layer 32 is in a range from 1 to 10 micrometers, or, more specifically, from 1 to 5 micrometers. With this configuration, necessary strength of the coating layer 32 is obtained, while enough slide efficiency of the piston 20 with the cylinder 12 is achieved.

A coating layer described above can be formed by several methods, for example by evaporating the materials to the surface of the core member 31 (i.e., vapor deposition method), or by applying a liquid composition including the materials to the surface of the core member 31. For the liquid composition, parylene resin (manufactured by Three Bond Co., Ltd) may be used.

Furthermore, the cross-sectional shape of the sealing member 30 may take any geometrical form other than a circle as shown in FIG. 2 and FIG. 3, including an ellipse, a rectangle, or a square.

The present invention is not limited to the embodiment of an operation button for an endoscope which is described herein with reference to the exemplary figures.

For example, each component of an operation button according to the present invention may alternatively be other members that may have equivalent functions. Also, optional members may be added to an operation button according to the present invention.

Further, while the sealing member described in the above embodiment is configured with a core member and a coating layer, there may be provided with one or more layers for a certain purpose (e.g., for enhancing sealing tightness) between the core member and the coating layer.

### ADDITIONAL EXPERIMENTAL EMBODIMENTS

Additional experimental embodiments according to the present invention are described below.

### Experimental Embodiment 1

A coating layer (average thickness: 5 micrometers) configured with poly-para-xylylene is formed to be an O-ring (i.e., a sealing member) by applying Xylene solution including poly-para-xylylene (polymerization degree: greater than 5,000) to the surface of a core member (diameter: 0.2 mm) which is configured with silicone rubber, and allowed to dry.

Then, the O-ring is embedded to an aspiration button (i.e., “OF-B120” manufactured by PENTAX Corp.), and installed in an endoscope (i.e., “EG-2930” manufactured by PENTAX Corp).

### Experimental Embodiment 2

An O-ring is created similarly to Experimental Embodiment 1 described above, except the poly-para-xylylene is altered to poly-monochloro-para-xylylene, and is embedded to an endoscope.

### COMPARATIVE SAMPLES

Comparative samples to the experimental embodiments are made as described below.

### Comparative Sample 1

An O-ring (diameter: 2.0 mm) configured with silicone rubber is created and embedded to an endoscope.

### Comparative Sample 2

An O-ring (diameter: 2.0 mm) configured with silicone rubber is created and embedded to an endoscope, with silicone oil as lubricant being applied.

### COMPARISON RESULT

The operation buttons in each Experimental Embodiment and each Comparative Sample described above were repeatedly pressed. Then, the suppress strengths of the first pressing operation and the 5,000^{th} pressing operation were measured for each endoscope by an autograph instrument manufactured by SHIMADZU Corp.

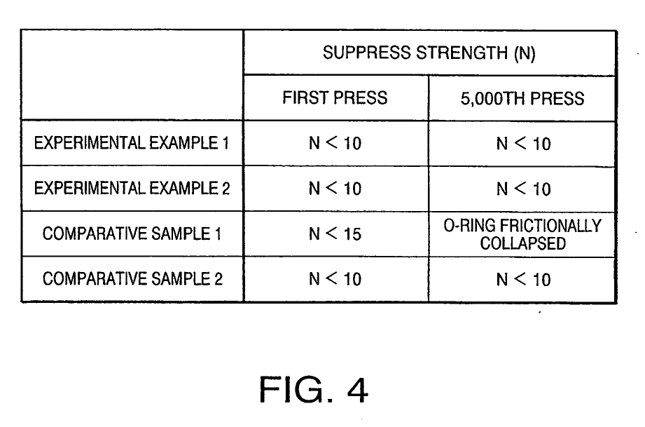

In addition, after every 10 pressing operation, silicone oil was applied to the surface of the O-ring of the operation button described in Comparative Sample 2. The measured results are shown in FIG. 4.

Each data indicates the averaged value measured with 10 O-rings.

As shown in FIG. 4, for the operation buttons described in Experimental Embodiment 1 and 2, it should be noted that no change is found in the suppress strengths between the first and 5,000^{th} pressing operations.

For the operation button described in Comparative Sample 1, on the other hand, the O-ring was frictionally collapsed before the 5,000^{th} pressing operation.

In addition, for the operation button described in Comparative Sample 2, also no change is found in the suppress strengths between the first and 5,000^{th} pressing operations. However, in order to maintain the equivalent suppress strength, it should be noted that application of silicone oil to the surface of the O-ring after every 10 pressing operation is required, which explicitly reduces operability. In this comparison result, the improved slide efficiency of the operation button and the eliminated inconvenience of applying lubricant can be found.

The present disclosure relates to the subject matter contained in Japanese Patent Application No. 2004-241620, filed on Aug. 20, 2004, which is expressly incorporated herein by reference in its entirety.

What is claimed is:

1. An operation button for an endoscope, comprising:
a cylinder having a lumen, a first flow channel and a second flow channel, the first flow channel and the second flow channel communicating with the lumen; and
a piston slidably provided inside the lumen of the cylinder, the piston being reciprocable between a first position and a second position, the first flow channel being prevented from communicating with the second flow channel when the piston is located at the first position, the first flow channel being allowed to communicate with the second flow channel when the piston is located at the second position; and
a sealing member provided at an outer periphery of the piston to provide secure airtightness between the cylinder and the piston,
wherein the sealing member includes:
a core member made of an elastic material; and
a coating layer allocated at an outer periphery of the core member, the coating layer being in immediate contact with an inner surface of the cylinder, the core member being mainly made of one of poly-para-xylylene and poly-para-xylylene derivatives.

2. An operation button for an endoscope according to claim 1,
wherein the polymerization degree of the one of the poly-para-xylylene and the poly-para-xylylene derivatives is greater than 5,000.

3. An operation button for an endoscope according to claim 1,
wherein the elastic material is mainly made of silicone rubber.

4. An operation button for an endoscope according to claim 1,
wherein an average thickness of the coating layer is in a range from 1 to 10 micrometers.

5. An operation button for an endoscope according to claim 1,
wherein the piston has a penetrative port, which is configured such that the penetrative port does not connect the first flow channel with the second flow channel when the piston is in the first position, and
wherein the penetrative port allows the first flow channel to communicate with the second flow channel through the penetrative port when the piston is in the second position.

6. An operation button for an endoscope according to claim 1,
wherein the cylinder and the piston are slidable with each other with requiring substantially no lubricant.

7. An endoscope including at least one operation button,
wherein the operation button comprises:
a cylinder having a lumen, a first flow channel and a second flow channel, the first flow channel and the second flow channel communicating with the lumen; and
a piston slidably provided inside the lumen of the cylinder, the piston being reciprocable between a first position and a second position, the first flow channel being prevented from communicating with the second flow channel when the piston is located at the first position, the first flow channel being allowed to communicate with the second flow channel when the piston is located at the second position; and
a sealing member provided at an outer periphery of the piston to provide secure airtightness between the cylinder and the piston,
wherein the sealing member includes a core member made of an elastic material, and a coating layer allocated at an outer periphery of the core member, the coating layer being in immediate contact with an inner surface of the cylinder, the core member being mainly made of one of poly-para-xylylene and poly-para-xylylene derivatives.
